(19) United States
(12) Patent Application Publication
(10) Pub. No.: US 2005/0204484 A1
(43) Pub. Date: Sep. 22, 2005

(54) Hair colorants

(75) Inventors: Markus Meder, Werbach (DE); Georg Schoefberger, Rhainfelden (CH)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/030,804

(22) Filed: Jan. 7, 2005

(30) Foreign Application Priority Data
Jan. 10, 2004 (DE) 102004001522.8

(51) Int. Cl. A61K 7/13

(52) U.S. Cl. 8/405

(57) ABSTRACT

The hair-coloring compositions described comprise one or more anthraquinone compounds of the formula I 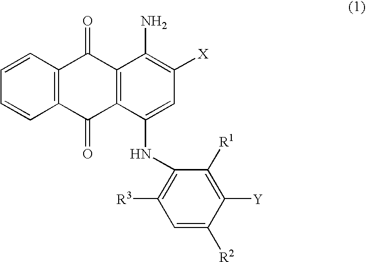
where R¹, R² and R³ each independently represent hydrogen, linear or branched alkyl having 1 to 22 carbon atoms, linear or branched alkenyl having 2 to 22 carbon atoms or N(R⁴)C(O)R⁵; R⁴ and R⁵ independently represent hydrogen or alkyl having 1 to 22 carbon atoms; X represents SO₃A¹ or OCH₂CH₂(OCH₂CH₂)ₓOCH₃; Y is H or SO₃A²; A¹ and A² each independently represent H⁺, Li⁺, Na⁺, K⁺, Mg²⁺/2, Ca²⁺/2, Al³⁺/3 or ⁺NR⁶R⁷R⁸R⁹; R⁶, R⁷, R⁸, R⁹ each independently represent hydrogen, alkyl having 1 to 22 carbon atoms or hydroxyalkyl having 2 to 10 carbon atoms, and x represents a number from 0 to 10; wherein the compounds of the formula I comprise exactly one group selected from SO₃A¹ and SO₃A².

This invention concerns compositions for dyeing and tinting human hair which comprise anthraquinone compounds.

Anthraquinones are an important class of direct dyes and are used for dyeing textiles in particular. Direct dyes as well as oxidative dyes have also been used for hair coloration to achieve a wide color gamut of yellow, orange, red, blue, green and violet colorations.

DE 33 13 337 describes anthraquinone compounds having two sulfonic acid groups and their use for dyeing fibers, in particular polyamides. However, these compounds have insufficient affinity for human hair and coloration is unsatisfactory.

U.S. Pat. No. 5,595,197 describes a process for dyeing human hair wherein a sulfo-containing dye has to be applied in a water vapor mixture at not less than 75° C. in order that good dyeing results may be obtained.

DE 202 13 695 U1 discloses a hair colorant comprising a blue anthraquinone dye combined with at least one further direct dye.

However, there continues to be a considerable need for direct dyes for coloration of keratin fiber, especially human hair, which are generally recognized as safe by toxicologists and make it possible to achieve colorations in the desired intensity under mild conditions. There is further a need for the colored hair obtained to possess good light, perming, acid and rub fastness. Hair colorations have to remain stable for at least 4 to 6 weeks without action of light, friction and chemical agents. In addition, direct dyes shall also be appliable in oxidative hair colorants.

We have found that, surprisingly, anthraquinone compounds of the formula I 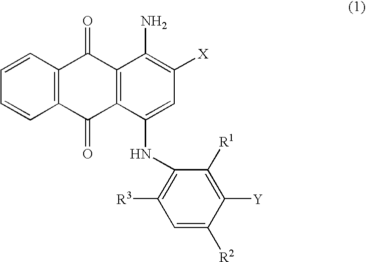
where
R¹, R² and R³ each independently represent hydrogen, linear or branched alkyl having 1 to 22, preferably 1 to 8 and more preferably 1 to 4 carbon atoms, linear or branched alkenyl having 2 to 22, preferably 2 to 8 and more preferably 2 to 4 carbon atoms or N(R⁴)C(O)R⁵,
R⁴ and R⁵ independently represent hydrogen or alkyl having 1 to 22, preferably 1 to 8 and more preferably 1 to 4 carbon atoms,
X represents SO₃A¹ or OCH₂CH₂(OCH₂CH₂)ₓOCH₃,
Y is H or SO₃A²,
A¹ and A² each independently represent H⁺, Li⁺, Na⁺, K⁺, Mg²⁺/2, Ca²⁺/2, Al³⁺/3 or ⁺NR⁶R⁷R⁸R⁹,
R⁶, R⁷, R⁸, R⁹ each independently represent hydrogen, alkyl having 1 to 22 and preferably 1 to 18 carbon atoms or hydroxyalkyl having 2 to 10 carbon atoms, and
X represents a number from 0 to 10, preferably 1 to 5 and more preferably 1,
   wherein the compounds of the formula I comprise exactly one group selected from SO₃A¹ and SO₃A²,
   are very useful as direct dyes for permanent and semipermanent hair colorants and can be used not only as color givers but, in combination with oxidative dye precursors, also as color tinge givers. We have further found that the compounds of the formula I are notable for high stability, good solubility in water, brilliant intensity of color, high shampooing stability and high light fastness. The dyes of formula I have beneficial physiological properties and are capable, on variation of the R¹ to R³ substituents for example, of providing hues ranging from blue to violet.

The present invention accordingly provides hair-coloring compositions comprising one or more anthraquinone compounds 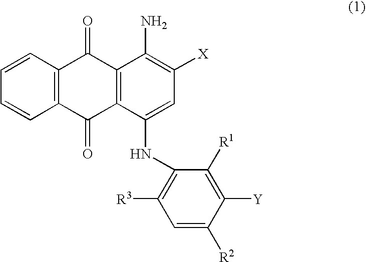
where
R¹, R² and R³ each independently represent hydrogen, linear or branched alkyl having 1 to 22, preferably 1 to 8 and more preferably 1 to 4 carbon atoms, linear or branched alkenyl having 2 to 22, preferably 2 to 8 and more preferably 2 to 4 carbon atoms or N(R⁴)C(O)R⁵,
R⁴ and R⁵ independently represent hydrogen or alkyl having 1 to 22, preferably 1 to 8 and more preferably 1 to 4 carbon atoms,
X represents SO₃A¹ or OCH₂CH₂(OCH₂CH₂)ₓOCH₃,
Y is H or SO₃A²,
A¹ and A² each independently represent H⁺, Li⁺, Na⁺, K⁺, Mg²⁺/2, Ca²⁺/2, Al³⁺/3 or ⁺NR⁶R⁷R⁸R⁹, preferably H⁺, Na⁺ or ⁺NR⁶R⁷R⁸R⁹,
R⁶, R⁷, R⁸, R⁹ each independently represent hydrogen, alkyl having 1 to 22 and preferably 1 to 18 carbon atoms or hydroxyalkyl having 2 to 10 carbon atoms, and
x represents a number from 0 to 10, preferably 1 to 5 and more preferably 1,
   wherein the compounds of the formula I comprise exactly one group selected from SO₃A¹ and SO₃A².

These hair-coloring compositions are especially useful for coloring human hair.

This invention further provides for the use of the compositions of the present invention for coloring keratin fiber and especially for coloring human hair.

In a preferred embodiment of the present invention the hair-coloring compositions comprise anthraquinone compounds of the formula I where
R¹, R² and R³ are the same, all representing hydrogen or all representing methyl,
X is SO₃A¹,
A¹ represents H⁺, Li⁺, Na⁺, K⁺, Mg²⁺/2, Ca²⁺/2, Al³⁺/3 or ⁺NR⁶R⁷R⁸R⁹, preferably H⁺, Na⁺ or ⁺NR⁶R⁷R⁸R⁹,
R⁶, R⁷, R⁸, R⁹ each independently represent hydrogen, alkyl having 1 to 22 and preferably 1 to 18 carbon atoms or hydroxyalkyl having 2 to 10 carbon atoms, and
Y represents H.

In a further preferred embodiment of the present invention the hair-coloring compositions comprise anthraquinone compounds of the formula I where
R¹ and R³ are hydrogen,
R² represents tert-butyl, n-butyl or —N(H)C(O)CH₃, and
X is SO₃A¹,
A¹ represents H⁺, Li⁺, Na⁺, K⁺, Mg²⁺/2, Ca²⁺/2, Al³⁺/3 or ⁺NR⁶R⁷R⁸R⁹, preferably H⁺, Na⁺ or ⁺NR⁶R⁷R⁸R⁹,
R⁶, R⁷, R⁸, R⁹ each independently represent hydrogen, alkyl having 1 to 22 and preferably 1 to 18 carbon atoms or hydroxyalkyl having 2 to 10 carbon atoms, and
Y represents H.

In a further preferred embodiment of the present invention the hair-coloring compositions comprise anthraquinone compounds of the formula I where
R¹, R² and R³ each represent methyl,
X represents OCH₂CH₂(OCH₂CH₂)ₓOCH₃,
x is 1,
Y represents SO₃A²,
A² represents H⁺, Li⁺, Na⁺, K⁺, Mg²⁺/2, Ca²⁺/2, Al³⁺/3 or ⁺NR⁶R⁷R⁸R⁹, preferably H⁺, Na⁺ or ⁺NR⁶R⁷R⁸R⁹, and
R⁶, R⁷, R⁸, R⁹ each independently represent hydrogen, alkyl having 1 to 22 and preferably 1 to 18 carbon atoms or hydroxyalkyl having 2 to 10 carbon atoms.

The compositions of the present invention can be used for permanent or semipermanent coloration of keratin fiber and preferably of human hair.

The compositions of the present invention may include the anthraquinone compounds of the formula I as sole coloring component as an individual substance or as a mixture.

In a further preferred embodiment of the present invention the compositions of the present invention, as well as the anthraquinone compounds of the formula I, comprise one or more further direct dyes to achieve further color tinges.

Preferred direct dyes are nitroaniline derivatives, such as 1-[(2-hydroxyethyl)amino]-2-nitrobenzene (Velsol™ Yellow 2), 4-hydroxypropylamino-3-nitrophenol (VelSol™ Red BN), 3-nitro-p-hydroxyethylaminophenol (Velsol™ Red 54), 4-hydroxyethylamino-3-nitroaniline (Velsol™ Red 3), N,N′-bis(hydroxyethyl)-2-nitro-p-phenylenediamines (Velsol™ Violet BS), N,N′,N′-tris(hydroxyethyl)-2-nitro-p-phenylenediamines (Velsol™ Blue 2), 4-(2′-hydroxyethyl)amino-3-nitrotoluene, 4-(2′-hydroxyethyl)amino-3-nitrobenzyl alcohol, 4-(2′-hydroxyethyl)amino-3-nitro-1-trifluoromethylbenzene, 4-(2′,3′-dihydroxypropyl)-amino-3-nitrochlorobenzene, 4-(2′-hydroxyethyl)amino-3-nitrobromobenzene and 4-(2′,3′-dihydroxypropyl)amino-3-nitrobromobenzene, nitrobenzene derivatives, for example 2-amino-4-nitrophenol, picramic acid, 1-[(2′-hydroxyethyl)amino]-2-amino-4-nitrobenzene, 2-nitro-4-[(2′-hydroxyethyl)amino]aniline, 4-bis[(2′-hydroxyethyl)amino]-1-methylamino-2-nitrobenzene, 2,5-bis[(2′-hydroxyethyl)amino]nitrobenzene, 2-(2′-hydroxyethyl)amino-4,6-dinitrophenol, 1-amino-4-(2′,3′-dihydroxypropyl)amino-2-nitro-5-chlorobenzene, but also triphenylmethane dyes, for example Basic Violet 1 (C.I. 42535), azo dyes, for example Acid Brown 4 (C.I. 14805), anthraquinone dyes not defined by the formula I, for example Disperse Blue 23 (C.I. 61545), Disperse Violet 4 (C.I. 61105), 1,4,5,8-tetraaminoanthraquinone and 1,4-diaminoanthraquinone, and also the direct dyes mentioned in DE 202 13 695 U1.

The pH value of non-oxidative hair-coloring compositions is preferably in the range from 2 to 7 and more preferably in the range from 4 to 6.

The anthraquinone compounds of the formula I can be prepared by methods familiar to one skilled in the art, for example by the methods described in CH 194 097, U.S. Pat. No. 2,117,569 and DE 642 726.

The hair-coloring compositions, in addition to the one or more anthraquinone compounds of the formula I, may comprise oxidative dye precursors and be used for permanent coloration of keratin fiber and especially of human hair.

In a further preferred embodiment of the invention the compositions of the present invention thus further comprise one or more oxidative dye precursors as well as the anthraquinone compounds of the formula I and if appropriate the further direct dyes.

The oxidative dye precursors are preferably p-phenylenediamines and p-aminophenols or their derivatives such as for example p-tolylenediamine, p-phenylenediamine, p-aminophenol, which can be combined with modifiers or couplers, such as m-phenylenediamine, resorcinol, m-aminophenol and derivatives thereof, to shade the coloration.

The level of anthraquinone compounds of the formula I present in the hair-coloring compositions of the present invention is preferably in the range from 0.001% to 15% by weight, more preferably in the range from 0.05% to 5% by weight and even more preferably in the range from 0.1% to 3% by weight, all based on the ready-produced hair-coloring compositions.

When the hair-coloring compositions of the present invention, as well as the anthraquinone compounds of the formula I, comprise one or more further direct dyes, the anthraquinone compounds of the formula I and the further direct dyes together will be present in the hair-coloring compositions of the present invention in amounts ranging preferably from 0.001% to 15% by weight, more preferably from 0.05% to 5% by weight and even more preferably from 0.1% to 3% by weight, all based on the ready-produced hair-coloring compositions.

When the hair-coloring compositions of the present invention comprise one or more oxidative dye precursors, the anthraquinone compounds of the formula I and the oxidative dye precursors together will be present in the hair-coloring compositions of the present invention in amounts ranging preferably from 0.01% to 15% by weight, more preferably from 0.1% to 5% by weight and even more preferably from 0.5% to 3% by weight, all based on the ready-produced hair-coloring compositions.

When the hair-coloring compositions of the present invention, as well as the anthraquinone compounds of the formula I, comprise one or more further direct dyes and one or more oxidative dye precursors, the anthraquinone compounds of the formula I, the further direct dyes and the oxidative dye precursors together will be present in the hair-coloring compositions of the present invention in amounts ranging preferably from 0.01% to 15% by weight, more preferably from 0.1% to 5% by weight and even more preferably from 0.5% to 3% by weight, all based on the ready-produced hair-coloring compositions.

Preferred oxidizing agents for developing the hair colorations include hydrogen peroxide and its addition compounds.

The pH value of these oxidative coloring compositions is preferably in the range from 2 to 11 and more preferably in the range from 7 to 10. An alkaline pH is preferably set using ammonia or organic amines, for example monoethanolamine or triethanolamine. An acidic pH is preferably set using citric acid, acetic acid, tartaric acid.

To enhance color intensity, the compositions of the present invention may comprise the carriers customary in cosmetic systems, preferably benzyl alcohol, vanillin (4-hydroxy-3-methoxybenzaldehyde), isovanillin, p-hydroxyanisole, 3-hydroxy-4-methoxybenzaldehyde, 2-phenoxyethanol, salicylaldehyde, 3,5-dihydroxybenzaldehyde, 3,4-dihydroxybenzaldehyde, 4-hydroxyphenylacetamide, methyl p-hydroxybenzoate, p-hydroxybenzaldehyde, m-cresol, hydroquinone monomethyl ether, o-fluorophenol, m-fluorophenol, p-fluorophenol, 2-(2′-hydroxyphenoxy)ethanol, 3,4-methylenedioxyphenol, resorcinol monomethyl ether, 3,4-dimethoxyphenol, 3-trifluoromethylphenol, resorcinol monoacetate, ethylvanillin, 2-thiophenethanol, butyl lactate and butyl glycolate.

The hair-coloring compositions of the present invention may advantageously comprise one or more pearlizing compounds, preferably fatty acid monoalkanolamides, fatty acid dialkanolamides, monoesters or diesters of alkylene glycol, especially esters of ethylene glycol and/or propylene glycol or oligomers thereof with higher fatty acids, for example palmitic acid, stearic acid or behenic acid or mixtures thereof, mono- or diesters of alkylene glycols with fatty acids, fatty acids and their metal salts, monoesters or polyesters of glycerol with carboxylic acids and ketosulfones of various kinds, more preferably ethylene glycol distearate and polyethylene glycol distearate having about 3 glycol units.

Glitter and luster effects due to the compositions of the present invention are preferably achieved by addition of mica, colored polyacrylic esters and mica, mica-iron oxide, mica-titania and by addition of pigments. Useful pigments include metal oxides, for example iron oxides, titania, ultramarine blue, and also pigments modified with cationic coating envelopes, these pigments being described in WO 00/12053 and EP 504 066.

The hair-coloring compositions of the present invention may include additives having a conditioning and color-preserving action on colored hair, examples being cationic and nonionic polymers.

Useful cationic polymers include not only the well-known quaternary cellulose derivatives of the Polymer JR type but also the compounds known under the INCI designation of Polyquaternium, especially Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, Polyquaternium 37 & mineral oil & PPG trideceth (Salcare® SC95), guarhydroxypropyl-triammonium chlorides, and also calcium alginate and ammonium alginate.

It is further possible to use aminosilicones, copolymers of diallylammonium salts and acrylamides; quaternized vinylpyrrolidone-vinylimidazole polymers, for example addition copolymers of vinylpyrrolidone and vinylimidazolinium methochloride which are available under the trade name of Luviquat; condensation products of polyglycols and amines; quaternized collagen polypeptides; quaternized wheat polypeptides; polyethyleneimines; cationic silicone polymers, for example amidomethicones; copolymers of adipic acid and dimethylaminohydroxypropyldiethylenetriamine; polyaminopolyamide and cationic chitin derivatives, for example chitosan, but also quaternized homo- and copolymers of dimethyldiallylammonium chloride which are commercially available under the trade name of Merquat; quaternary vinylpyrrolidone copolymers, especially with dialkylaminoalkyl (meth)acrylates which are known under the name of Gafquat, polyamino-polyamide derivatives, for example copolymers of adipic acid and dimethylaminohydroxypropyldiethylenetriamine which are marketed under the name of Cartaretine F, and also bisquaternary long-chain ammonium compounds of the urea structure described in U.S. Pat. No. 4,157,388 which are known under the trade name of Mirapol A 15.

Nonionic polymers can be used instead of cationic polymers or in combination with the same. Useful nonionic polymers include in particular vinylpyrrolidone homo- and copolymers, especially polyvinylpyrrolidone itself, copolymers of vinylpyrrolidone and vinyl acetate or terpolymers of vinylpyrrolidone, vinyl acetate and vinyl propionate which are for example marketed by BASF under the trade name of Luviskol.

It is also possible to use (co)polymers of the various acrylic and methacrylic esters, acrylamide and methacrylamide, for example polyacrylamide having molecular weights of above 100 000, or dimethylhydantoin-formaldehyde resins.

Similarly suitable are amphoteric polymers, for example the addition copolymers of N-octylacrylamide, N-butylaminoethyl methacrylate and acrylic acid which are marketed under the designation of Amphomer.

Natural polymers, such as chitosan or chitosan derivatives, can also be used.

The amounts in which the abovementioned polymers are used are preferably in the range from 0.1% to 5% by weight, more preferably in the range from 0.25% to 2.5% by weight and even more preferably in the range from 0.5% to 1.5% by weight, based on the overall makeup of the hair-coloring compositions.

The hair-coloring compositions of the present invention may include further additives, examples being fats and oils, fatty alcohols, emulsifiers, wetting agents, thickeners, softeners, pH regulators, solvents, solubilizers, preservatives or perfumes.

Useful fats and oils, including waxes, include for example natural oils such as avocado oil, coconut oil, palm kernel oil, sesame oil, peanut oil, sperm oil, sunflower oil, almond oil, peach kernel oil, wheat germ oil, macadamia nut oil, evening primrose oil, jojoba oil, castor oil or else olive or soybean oil, lanolin and its derivatives, and also mineral oils such as paraffin oil and white petroleum jelly.

Synthetic oils and waxes useful in the compositions of the present invention are for example silicone oils or polyethylene glycols.

Further suitable hydrophobic components for the hair-coloring compositions of the present invention are in particular fatty alcohols, preferably those having 8 to 22 carbon atoms, examples being myristyl alcohol, cetyl alcohol, stearyl alcohol, waxy alcohols and fatty acid esters such as isopropyl myristate, isopropyl palmitate, isopropyl stearate, isopropyl isostearate, oleyl oleate, isocetyl stearate, hexyl laurate, dibutyl adipate, dioctyl adipate, myristyl myristate, oleyl erucate, polyethylene glycol- and polyethylene glycol fatty acid esters such as PEG-7-glyceryl cocoate or cetyl palmitate.

The total amount of these hydrophobic components present in the compositions of the present invention is preferably in the range from 0.5% to 10% by weight, more preferably in the range from 1% to 7.5% by weight and even more preferably in the range from 1.5% to 5% by weight, reckoned on total composition.

Solvents used are preferably water, lower aliphatic alcohols, examples being ethanol, propanol, isopropanol, glycerol or glycols such as ethylene glycol, propylene glycol or else glycol ethers.

Available for use as wetting agents and emulsifiers are anionic, cationic, amphoteric or nonionic surface-active substances such as fatty alcohol sulfates, fatty alcohol ether sulfates, alkylsulfonates, alkylbenzene sulfates, alkyltrimethylammonium salts, alkylbetaines, ethoxylated fatty alcohols, ethoxylated nonylphenols, fatty acid alkanolamides and ethoxylated fatty acid esters.

Higher fatty alcohols, bentonite, starch, polyacrylic acid, cellulose derivatives such as carboxymethylcellulose, alginates, white petroleum jelly, paraffin oil and fatty acids can be used for thickening the compositions.

Wetting agents and emulsifiers are used in concentrations of 0.5% to 30% by weight, based on the ready-produced coloring compositions, whereas thickeners can be present in an amount of 0.1% by weight to 25% by weight.

Conditioners are for example lanolin derivatives, cholesterol, pantothenic acid, which may comprise from 0.1% to 5% by weight of the hair-treating compositions of the present invention.

When those of the aforementioned preparations which require the addition of an oxidizing agent come to be used the addition of an oxidizing agent is accomplished in known manner, by mixing the hair-coloring compositions with the oxidizing agent prior to the treatment.

The present invention's coloring composition comprising one or more anthraquinone compounds of the formula I or a mixture of anthraquinone compounds of the formula I with further direct dyes, if appropriate combined with oxidative dye precursors, is applied to hair in an amount of the mixture that is sufficient to color the hair, this amount being in general about 50 to 150 ml, and is left on the hair for about 1 to 45 minutes at 20 to 50° C. and preferably for 15 to 30 minutes at about 40° C. This is followed by rinsing with water, if appropriate additionally with the aqueous solution of a weak organic acid, and drying. Examples of useful weakly organic acids are acetic acid, citric acid, tartaric acid and the like.

The form in which the hair-coloring compositions of the present invention are prepared is preferably a solution, especially an aqueous or an aqueous alcoholic solution, an emulsion, a dispersion, a cream, a gel or an aerosol foam or spray.

The examples which follow illustrate the invention.

### EXAMPLES

### Example 1

Colorants 1 to 9 were prepared with the hereinbelow indicated composition.

| Sodium lauryl ether sulfate | 7 | g |
|---|---|---|
| Dye as per table 1 | 0.5 | g |
| Ethanol | 10 | g |
| NaCl | q.s. | |
| Water | ad 100 | g |

### Preparation

The components were mixed at room temperature. The pH was set to about 5 with citric acid or NaOH. The dyes and dye mixtures used in colorants 1-9 are listed in table 1. All dyes listed in table 1 are commercially available.

TABLE 1
Dyes and dye mixtures used in colorants 1-9

| Dye | Colorant | | | | | | | | |
|---|---|---|---|---|---|---|---|---|---|
| (C.I. name) | 1 | 2 | 3 | 4 | 5 | 6 | 7 | 8 | 9 |
| Velsol ™ Blue 25 | 0.5 g | — | — | — | 0.3 g | 0.3 g | — | — | — |
| (Acid Blue 25) | | | | | | | | | |
| Velsol ™ Blue 129 | — | 0.5 g | — | — | — | — | 0.3 g | — | — |
| (Acid Blue 129) | | | | | | | | | |
| Velsol ™ Blue 230 | — | — | 0.5 g | — | — | — | 0.1 g | — | — |
| (Acid Blue 230) | | | | | | | | | |
| Velsol ™ Blue 40 | — | — | — | 0.5 g | — | — | — | 0.2 g | — |
| (Acid Blue 40) | | | | | | | | | |
| Velsol ™ Violet 126 | — | — | — | — | 0.2 g | — | — | 0.2 g | 0.5 g |
| (Acid Violet 126) | | | | | | | | | |
| Velsol ™ Violet BS | — | — | — | — | — | 0.1 g | — | — | — |
| (−) | | | | | | | | | |
| Velsol ™ Red BN | — | — | — | — | — | 0.1 g | 0.1 g | 0.1 g | — |
| (−) | | | | | | | | | |

### Chemical Identification of Dyes

| | |
|---|---|
| Velsol ™ Blue 25 | Formula I, R¹ = R² = R³ = H, X = SO₃H, Y = H |
| Velsol ™ Blue | Formula I, R¹ = R² = R³ = CH₃, X = SO₃H, Y = H |
| 129 | |
| Velsol ™ Blue | Formula I, R¹ = R³ = H, R₂ = tert-butyl, X = SO₃H, |
| 230 | Y = H |
| Velsol ™ Blue 40 | Formula I, R¹ = R³ = H, R² = N(H)C(O)CH₃, |
| | X = SO₃H, Y = H |
| Velsol ™ Violet | Formula I, R¹ = R² = R³ = CH₃, |
| 126 | X = OCH₂CH₂(OCH₂CH₂)ₓOCH₃, x = 1, Y = SO₃H |
| Velsol ™ Violet | N,N′-Bis(2-hydroxyethyl)-2-nitro-p- |
| BS | phenylenediamine |
| Velsol ™ Red | 4-Hydroxypropylamino-3-nitrophenol |
| BN | |

### EXAMPLE 2

Colorants 1-9 of Example 1 were used to color strands of Caucasian hair which had been bleached blond. The results are listed in table 2.

TABLE 2
Coloration of blond bleached strands of
Caucasian hair with colorants 1 to 9

| Colorant | Color of colored strands |
|---|---|
| 1 | blue |
| 2 | grayish green |
| 3 | greenish |
| 4 | greenish |
| 5 | bluish gray |
| 6 | aubergine |
| 7 | bordeaux |
| 8 | copper |
| 9 | ash blond |

### EXAMPLE 3

Colorants 1 to 5 of Example 1 were tested for shampooing stability. First the well-known L and E metrics were determined using appropriately colored strands. Thereafter, the colored strands were washed four times with a commercially available shampoo at about 35° C. and then remeasured for L and E before the change in lightness (DL) and total color change (DE) were determined by subtraction. The results are listed in table 3.

TABLE 3
DL and DE values for colorants 1-5

| Colorant | DL | DE |
|---|---|---|
| 1 | 2.62 | 3.44 |
| 2 | 3.12 | 7.05 |
| 3 | 3.19 | 5.64 |
| 4 | 0.2 | 1.97 |
| 5 | 2.07 | 4.8 |

The DL and DE values were determined according to the CIE L *a *b, D65/10° method using a Datacolor Mercury 2000 colorimeter.

The results of table 3 show that the anthraquinone-based colorants tested are notable for high shampooing stability.

Formulation examples (all percentages by weight)

Tinting shampoo

| | | | |
|---|---|---|---|
| A | GENAGEN ® CAB 818 | (Clariant) | 7.0% |
| | Cocoamidopropyl Betaine | | |
| | VELSOL ™ Blue 25 | (Clariant) | 0.5% |
| | Water | | 30.0% |
| B | GENAPOL ® T 500 P | Clariant) | 0.5% |
| | Ceteareth-50 | | |
| | Water | | ad 100.0% |
| C | GENAPOL ® LRO liquid | (Clariant) | 30.0% |
| | C_{12/14}-Alkyl diglycol ether sulfate, Na salt | | |
| | GENAGEN ® LAA | (Clariant) | 3.0% |
| | Lauroamphoacetate, Na salts | | |
| | GENAMIN ® CTAC | (Clariant) | 1.0% |
| | Cetrimonium chloride | | |
| | Ethylenediaminetetraacetate, Na salt 0.1% | | |
| | NIPAGUARD ® DMDMH | (Clariant) | 0.3% |
| | DMDM Hydantoin | | |
| | [1,3-Bis(Hydroxymethyl)-5,5- | | |
| | dimethylimidazolidine-2,4-dione] | | |
| | GENAPOL ® PDB | (Clariant) | 3.0% |
| | Glycol Distearate and Laureth-4 and | | |
| | Cocoamidopropyl Betaine | | |
| | Potassium phosphates | | 1.5% |
| D | Citric acid | | q.s. |

Method of making:
I Dissolve components A with stirring
II Mix components B and dissolve with stirring and gentle heating
III Cool B to about 35° C.; then add components C to B
IV Stir components A into B and C

V Set pH 5.5 with component D

Setting foam with tint

| | | | |
|---|---|---|---|
| A | Water | | q.s. |
| | GENAPOL ® C 100 | (Clariant) | 1.0% |
| | Coceth-10 | | |
| | Polyquaternium-10 | | 0.1% |
| | GENAMIN ® CTAC | (Clariant) | 0.2% |
| | Cetriumonium Chloride | | |
| | GENAMIN ® KDMP | (Clariant) | 0.2% |
| | Behentrimonium Chloride | | |
| B | Ethanol | | 3.0% |
| | Water | | 5.0% |
| | DIAFORMER ® Z-651 | (Clariant) | 3.5% |
| | Acrylates/Lauryl Acrylate/Stearyl Acrylate/ | | |
| | Ethylamine Oxide Methacrylate | | |
| | Copolymer | | |
| | VELSOL ™ Violet 126 | (Clariant) | 0.4% |
| C | Citric acid | | q.s. |

Method of making:
I Heat components A to about 75° C.
II Dissolve components B with stirring
III Cool A down to about 35° C.
IV Add B to A
V Set pH 4.0 with C

1. A hair-coloring composition comprising one or more anthraquinone dyes of the formula I 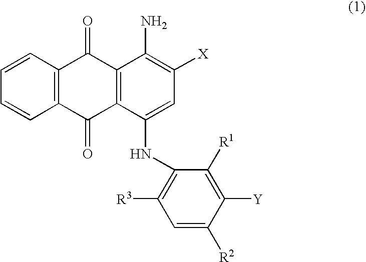
where
R¹, R² and R³ each independently represent hydrogen, linear or branched alkyl having 1 to 22 carbon atoms, linear or branched alkenyl having 2 to 22 carbon atoms or N(R⁴)C(O)R⁵,
R⁴ and R⁵ independently represent hydrogen or alkyl having 1 to 22 carbon atoms,
X represents SO₃A¹ or OCH₂CH₂(OCH₂CH₂)ₓOCH₃,
Y is H or SO₃A²,
A¹ and A² each independently represent H⁺, Li⁺, Na⁺, K⁺, Mg²⁺/2, Ca²⁺/2, Al³⁺/3 or ³⁰ NR⁶, R⁷, R⁸, R⁹,
R⁶, R⁷, R⁸, R⁹ each independently represent hydrogen, alkyl having 1 to 22 or hydroxyalkyl having 2 to 10 carbon atoms, and
x represents a number from 0 to 10, wherein the compounds of the formula I comprise exactly one group selected from SO₃A¹ and SO₃A².

2. The hair-coloring composition according to claim 1 wherein
R¹, R ²and R³ are the same, all representing hydrogen or all representing methyl,
X is SO₃A¹,
A¹ represents H⁺, Li⁺, Na⁺, K⁺, Mg²⁺/2, Ca²⁺/2, Al³⁺/3 or ⁺NR⁶R⁷R⁸R⁹,
R⁶, R⁷, R⁸, R⁹ each independently represent hydrogen, alkyl having 1 to 22 carbon atoms or hydroxyalkyl having 2 to 10 carbon atoms, and
Y represents H.

3. The hair-coloring composition according to claim 1 wherein
R¹ and R³ are hydrogen,
R² represents tert-butyl, n-butyl or —N(H)C(O)CH₃, and
X is SO₃A¹,
A¹ represents H⁺, Li⁺, Na⁺, K⁺, Mg²⁺/2, Ca²⁺/2, Al³⁺/3 or ⁺NR⁶R⁷R⁸R⁹,
R⁶, R⁷, R⁸, R⁹ each independently represent hydrogen, alkyl having 1 to 22 carbon atoms or hydroxyalkyl having 2 to 10 carbon atoms, and
Y represents H.

4. The hair-coloring composition according to claim 1 wherein
R¹, R² and R³ each represent methyl,
X represents OCH₂CH₂(OCH₂CH₂)ₓOCH₃,
x is 1,
Y represents SO₃A²,
A² represents H⁺, Li⁺, Na⁺, K⁺, Mg²⁺/2, Ca²⁺/2, Al³⁺/3 or ⁺NR⁶R⁷R⁸R⁹, and
R⁶, R⁷, R⁸, R⁹ each independently represent hydrogen, alkyl having 1 to 22 carbon atoms or hydroxyalkyl having 2 to 10 carbon atoms.

5. The hair-coloring composition of claim 1 which comprises one or more further direct dyes as well as one or more anthraquinone compounds of the formula I.

6. The hair-coloring composition of claim 1 which further comprises one or more oxidative dye precursors.

7. The hair-coloring composition of claim 1 which further comprises one or more carriers.

8. The hair-coloring composition of claim 1 which further comprises one or more pearlizing compounds.

9. The hair-coloring composition of claim 1 which further comprises one or more compounds having glitter or luster effects.

10. The hair-coloring composition of claim 1 which further comprises one or more compounds having conditioning and color-preserving effects.

11. The hair-coloring composition of claim 1 which comprises 0.001% to 15% by weight of anthraquinone compounds of formula I, based on the hair-coloring composition.

12. The hair-coloring composition of claim 1, which is in a form selected from the group consisting of a solution, an emulsion, a dispersion, a cream, a gel, an aerosol foam, and a spray.

13. A method for coloring keratin fiber, said method comprising contacting the keratin fiber with the hair coloring composition of claim 1.

14. The hair coloring composition of claim 1, wherein R¹, R² and R³ each represent hydrogen, linear or branched alkyl having 1 to 8 carbon atoms, linear or branched alkenyl having 2 to 8 carbon atoms or N(R⁴)C(O)R⁵.

15. The hair coloring composition of claim 1, wherein R¹, R² and R³ each represent hydrogen, linear or branched alkyl having 1 to 4 carbon atoms, linear or branched alkenyl having 2 to 4 carbon atoms or N(R⁴)C(O)R⁵.

16. The hair coloring composition of claim 1, wherein R⁴ and R⁵ independently represent hydrogen or alkyl having 1 to 8 carbon atoms.

17. The hair coloring composition of claim 1, wherein R⁴ and R⁵ independently represent hydrogen or alkyl having 1 to 4 carbon atoms.

18. The hair coloring composition of claim 1, wherein A¹ and A² each independently represent H⁺, Na⁺ or ⁺NR⁶R⁷R⁸R⁹.

19. The hair coloring composition of claim 1, wherein R⁶, R⁷, R⁸, R⁹ each independently represent hydrogen, alkyl having 1 to 18 carbon atoms or hydroxyalkyl having 2 to 10 carbon atoms.

20. The hair coloring composition of claim 1, wherein x is a number from 1 to 5.

21. The hair coloring composition of claim 1, wherein x is 1.

22. The hair coloring composition of claim 2, wherein A¹ represents H⁺, Na⁺ or ⁺NR⁶R⁷R⁸R⁹ and R⁶, R⁷, R⁸, R⁹ each independently represent hydrogen, alkyl having 1 to 18 carbon atoms or hydroxyalkyl having 2 to 10 carbon atoms.

23. The hair coloring composition of claim 3, wherein A¹ represents H⁺, Na⁺ or ⁺NR⁶R⁷R⁸R⁹ and R⁶, R⁷, R⁸, R⁹ each independently represent hydrogen, alkyl having 1 to 18 carbon atoms or hydroxyalkyl having 2 to 10 carbon atoms.

24. The hair coloring composition of claim 4, wherein A² represents H⁺, Na⁺ or ⁺NR⁶R⁷R⁸R⁹ and R⁶, R⁷, R⁸, R⁹ each independently represent hydrogen, alkyl having 1 to 18 carbon atoms or hydroxyalkyl having 2 to 10 carbon atoms.

25. The hair coloring composition of claim 12, which is in the form of an aqueous or aqueous alcoholic solution.

26. The method of claim 13, wherein the keratin fiber is human hair.
